(19) United States
(12) Patent Application Publication
(10) Pub. No.: US 2009/0269801 A1
(43) Pub. Date: Oct. 29, 2009

(54) Vector to Induce Expression of Recombinant Proteins under Anoxic or Microaerobic Conditions

(76) Inventors: Anne M. Gardner, Dayton, OH (US); Paul R. Gardner, Dayton, OH (US)

Correspondence Address: THOMPSON HINE L.L.P.;Intellectual Property Group, P.O. BOX 8801, DAYTON, OH, 45401-8801, US

(21) Appl. No.: 11/795,394

(22) PCT Filed: Jan. 17, 2006
(86) PCT No.: PCT/US06/01543

§ 371 Date: Mar. 3, 2009

Related U.S. Application Data
(60) Provisional application No. 60/645,251, filed on Jan. 19, 2005.

(51) Int. Cl. C12P 21/00; C12N 15/00; C12P 19/34

(52) U.S. Cl. 435/69.1; 435/320.1; 435/91.4

(57) ABSTRACT

A protein expression vector that expresses large quantities of recombinant proteins under anoxic or microaerobic conditions by inducing expression with nitrate. The vector backbone is pUC19 and protein expression is driven by the E. coli flavohemoglobin promoter, which is inducible by nitrate, nitrite, or nitric oxide under conditions of low oxygen. The Nde1 site of pUC19 has been destroyed by filling in with Klenow fragment and religating the vector. An Nde1 site in the promoter provides an in-frame start methionine and a standard polylinker is available for ease of subcloning. The vector is named pANX for Plasmid ANaerobic eXpression.

The U.S. Government has a paid-up license in this invention and the right in limited circumstances to require the patent owner to license others on reasonable terms as provided for by the terms of Grant No. GM65090 awarded by the National Institutes of Health.

### FIELD OF THE INVENTION

The invention is directed to vectors used to express recombinant proteins.

### BACKGROUND

Many commercially important protein products are synthesized in Escherichia coli (E. coli). The two major goals targeted in cell-based recombinant protein production, in order to maximize protein production, are high cell density and high-level gene expression. Culture performance is optimized when these two goals are simultaneously met. High cell density can be obtained in culture using fed-batch cultivation, in which concentrated medium is gradually fed into a bioreactor, as described in Riesenberg, D., and R. Guthke, 1999, High-cell-density cultivation of microorganisms. Appl. Microbiol. Biotechnol. 51:422-430, the disclosure of which is incorporated by reference herein in its entirety. High-level protein production in E. coli requires strong, inducible promoters that are capable of initiating rapid protein synthesis and that can quickly produce large amounts of protein after induction. Inducible expression vectors including lac, trp, tac, and phage T7 promoters have been successfully used as described in Makrides, S.C., 1996, Strategies for achieving high-level expression of genes in Escherichia coli. Microbiol Rev. 60:512-538, the disclosure of which is incorporated by reference herein in its entirety. These vectors rely on aeration throughout the growth and induction phases of culture. Inadequate oxygenation results in poor cell growth and reduced protein yields. Maintaining proper aeration once a culture is dense is difficult and expensive.

Aeration may lead to other problems as well. For example, overexpression of foreign proteins in E. coli frequently results in the formation of insoluble inclusion bodies composed of inactive protein as described in Villaverde, A., and M. M. Carrio, 2003, Protein aggregation in recombinant bacteria: biological role of inclusion bodies. Biotechnol. Lett. 25:1385-1395, the disclosure of which is incorporated by reference herein in its entirety. The mechanism of inclusion body formation is not completely understood. It is believed that the overexpressed gene products cannot be correctly folded and processed to achieve the native protein structure. Further, it is believed that aeration is a factor in inclusion body formation. For example, NorR is a transcription factor that regulates the expression of the anoxic NorVW nitric oxide reductase in E. coli. NorR is normally expressed in low levels. Attempts to overexpress the protein by conventional methods of induction (under conditions of aeration) were unsuccessful due to the fact that the protein contains an oxygen labile heme co-factor. Thus, overexpression of the protein led to formation of insoluble inclusion bodies, substoichiometric heme content, and no active, purifiable protein. Attempts to reduce the formation of inclusion bodies, including increasing the expression of chaparones, and processing proteins, have met with limited success. (See Goloubinoff, P., A. A. Gatenby, and G. H. Lorimer, 1989, GroE heat-shock proteins promote assembly of foreign prokaryotic ribulose bisphosphate carboxylase oligomers in Escherichia coli. Nature. 337:44-47, and Ostermeier, M., K. DeSutter, and G. Georgiou, 1996, Euraryatic protein disulfide isomerase complements Eschrichia coli dsbA mutants and increases the yield of a heterologous secreted protein with disulfide bonds. J Biol. Chem. 271:10616-10622, the disclosures of which are incorporated by reference herein in their entireties.)

As can be seen, one drawback to the use of vectors that rely on aeration is that many proteins react unfavorably with molecular oxygen, and thus cannot be expressed in a usable form under conditions of high aeration. As examples of oxygen-reactive proteins, nitric oxide dioxygenases (NODs) catalyze the reaction NO+O₂+e⁻→NO₃⁻, but can also release toxic superoxide radical and hydrogen peroxide as byproducts. NODs normally provide a free radical defense mechanism by detoxifying nitric oxide (NO). NO is a radical that builds up to toxic amounts when induced by responses to infections, foreign bodies, or tissue injury. At low levels, NO acts as a signal and controls diverse physiological processes including vasotension and O₂ delivery to tissues. NOD protects diverse cells and organisms from NO poisoning, growth inhibition and killing. NOD also modulates NO signaling pathways controlling vasorelaxation.

The structure and enzymatic function of NOD from E. coli, a flavohemoglobin-type NOD, has been reported (Gardner et al., Proc. Natl. Acad. Sci. USA 95, 13089 (1998) which is expressly incorporated by reference herein in its entirety). The reaction steps for flavohemoglobin-catalyzed NO dioxygenation incorporate the NADPH, FAD, and O₂ dependence, as well as other features, of the mammalian hemoglobin. Mammalian NOD is a microsomal cytochrome P450 oxidoreductase (EC 1.6.2.4)-driven heme-dependent enzyme (Hallstrom et al. Free Radic. Biol. Med. 37(2) (2004)), which is expressly incorporated by reference herein in its entirety). NODs are examples of proteins that react with oxygen to form toxic superoxide and hydrogen peroxide.

Uses for NOD and other proteins are thus desirable. However, the expression of NOD and other oxygen-sensitive or oxygen-reactive proteins is difficult under conditions of high aeration. Thus, a protein expression vector that expresses large quantities of recombinant proteins under anoxic or microaerobic conditions is desirable.

### SUMMARY OF THE INVENTION

In one embodiment, the present invention includes an expression vector for the production of proteins, such as oxygen-sensitive, oxygen-radical-generating, or oxygen-reactive proteins, in E. coli The vector is referred to herein as pANX (Plasmid ANaerobic eXpression). Use of the pANX vector for the expression of proteins occurs under anoxic or microaerobic conditions. Nitrate may be used as both the respiratory substrate to support a high level of growth under low aeration or anoxic conditions and as the inducer of expression from a nitrate inducible promoter from the hmp gene (the E. coli flavohemoglobin promoter). Protein expression may also be induced by nitrite or nitric oxide. Using the pANX vector, the induction and purification of protein, such as the heme-containing transcription factor NorR in substantial quantities, can be achieved. Of the soluble protein produced using the pANX vector of the present invention, about 20%-30% was heme-containing NorR. This results from the anoxic induction conditions used to induce protein expression from pANX. The pANX vector can also be used to overexpress other proteins, including, but not limited to, the mammalian protein heme oxygenase 1 (HOX-1), the oxygen-sensitive NorV/NorW nitric oxide reductase, C. albicans flavohemoglobin, and several site-directed mutants of E. coli NOD. Proteins such as flavohemoglobin, which can be produced in a soluble form by conventional expression plasmids, are reliably induced in the pANX vector as well.

Expression from the vector of the present invention can be induced by nitrate under anoxic or microaerobic conditions, because the hmp promoter used in the vector is nitrate inducible. (Poole et al., Nitric Oxide, Nitrite, and Fnr Regulation of hmp (Flavohemoglobin) Gene Expression in Escherichia coli K-12, Journal of Bacteriology, (September 1996), pp. 5487-5492, which is expressly incorporated by reference herein in its entirety). As a result, the present invention eliminates the need to maintain constant aeration, and thus is simpler and less expensive.

The pANX vector includes a modified pUC19 vector and a nitrate-inducible promoter. For example, protein expression is driven by the E. coli flavohemoglobin promoter, which is inducible by nitrate under anaerobic and microaerobic conditions. An Nde1 site provides an in-frame start methionine and a polylinker is available for subcloning.

Another embodiment of the invention is a protein expression vector adapted to express recombinant proteins under anoxic conditions.

The protein expressed may include, but is not limited to, NOD, NorR, NorV, or HOX-1.

Another embodiment of the invention is a protein expression vector adapted to express recombinant proteins under microaerobic conditions. The protein expressed may include, but is not limited to, NOD, NorR, NorV, or HOX-1.

Another embodiment of the invention is a method of expressing a recombinant protein under anoxic conditions. The method includes introducing a gene coding for a particular protein into a vector, and expressing the protein in a host cell by inducing expression with nitrate, nitrite, or nitric oxide.

Another embodiment of the invention is a method of expressing a recombinant protein under microaerobic conditions. The method includes introducing a gene coding for a particular protein into a vector, and expressing the protein in a host cell by inducing expression with nitrate.

Another embodiment of the invention is a method of expressing a recombinant protein under microaerobic conditions. The method includes introducing a gene coding for a particular protein into a vector, and expressing the protein in a host cell by inducing expression with nitrite or nitric oxide.

Another embodiment of the invention is a protein expression vector including a nitrate-inducible promoter for anoxic or microaerobic expression of proteins.

Another embodiment of the invention is a method for expressing proteins in a vector including the use of a nitrate-inducible promoter for anoxic or microaerobic expression of proteins. In one embodiment, the hmp promoter is used.

Another embodiment of the invention is a method for expressing oxygen-sensitive, oxygen-radical-generating, or oxygen-reactive proteins in a host by providing to a pUC vector a nitrite-inducible promoter with an insert of DNA sufficient to encode a functional oxygen-sensitive, oxygen-radical-generating, or oxygen-reactive protein, and providing nitrite to induce the promoter, thereby expressing the oxygen-sensitive, oxygen-radical-generating, or oxygen-reactive protein.

Another embodiment of the invention is a pUC plasmid containing an insert of a promoter inducible by at least one of nitrate, nitrite, or nitric oxide, thereby forming a modified pUC plasmid. This modified pUC plasmid induces expression of an inserted gene encoding oxygen-sensitive, oxygen-reactive, or oxygen-radical-generating proteins under at least microaerobic conditions.

Another embodiment of the invention is an isolated inducible promoter including a fragment of the 812 bp E. coli flavohemoglobin upstream region operably linked to a nitric oxide dioxygenase coding region. The promoter fragment is sufficient to induce expression of a gene encoded therein by at least one of nitrate, nitrite, or nitric oxide.

Another embodiment of the invention is a method of generating a nitrate inducible expression vector by inserting an expression-inducing fragment of an E. coli flavohemoglobin upstream region and coding region for nitric oxide dioxygenase into a pUC vector, thereby forming a modified pANX vector; ligating into the pANX vector a DNA sequence encoding at least one of an oxygen-sensitive, oxygen-radical-generating, or oxygen-reactive protein; transforming a bacterial host with the ligated pANX vector; and selecting colonies that contain the ligated pANX vector.

Another embodiment of the invention is an E. coli transformed with the pANX vector.

These and other advantages will be apparent in light of the following figures and detailed description.

### BRIEF DESCRIPTION OF THE DRAWINGS

FIG. 1 is a diagram of an embodiment of the pANX vector;

FIG. 2A shows the induction of NOD expression using the pANX vector, with Lane 1 depicting molecular weight markers in kD, Lane 2 depicting pANX, and Lane 3 depicting pANXNOD;

FIG. 2B shows the induction of NorR expression using the pANX vector, with Lane 1 depicting pANX, Lane 2 depicting pANXNORR, and Lane 3 depicting molecular weight markers in kD;

FIG. 2C shows the induction of NorV expression using the pANX vector, with Lane 1 depicting molecular weight markers in kD, Lane 2 depicting pANX, and Lane 3 depicting pANXNORV; and

FIG. 2D shows the induction of HOX-1 expression using the pANX vector, with Lane 1 depicting pANX, Lane 2 depicting pANXHOX-1, and Lane 3 depicting molecular weight markers in kD.

### DETAILED DESCRIPTION

The present invention includes an expression vector for the production of oxygen-sensitive or reactive-oxygen (e.g., superoxide radical) generating proteins in E. coli. The vector is referred to as pANX herein (Plasmid ANaerobic eXpression). Use of the pANX vector for the expression of proteins occurs under anoxic (i.e., anaerobic) or microaerobic conditions. “Anoxic” and “anaerobic” refer to conditions in which oxygen is absent. “Microaerobic” refers to conditions with suboptimal oxygen. Nitrate can be used as both the respiratory substrate to support a high level of growth under low aeration (i.e., microaerobic) or anoxic conditions and as the inducer of expression from a nitrate inducible promoter. An example of such a promoter is from the hmp gene (the E. coli flavohemoglobin promoter).

The protein expression vector of the present invention is a plasmid. Plasmids are naturally occurring accessory chromosomes in a host, i.e., naturally occurring circular DNA molecules that carry genes for antibiotic inactivation, toxin production, and/or breakdown of natural products. These plasmids replicate independently of the host chromosome.

As is well known to those skilled in the art, particular plasmids to express a target protein may be created through modification of existing plasmids. In general, plasmids are modified by cleaving the circular DNA using endonucleases, and DNA fragments can then be inserted. A particular organism, such as E. coli, takes up the plasmid. Insertion of DNA from another source into the cloning site is confirmed by PCR (polymerase chain reaction) or DNA sequencing. E. coli provides the transcriptional and translational machinery to express the protein encoded in the insert of the plasmid.

The pANX protein expression vector described herein is a modified pUC19 vector. The base plasmid vector pUC19 contains a polylinker with unique cloning sites for multiple restriction nucleases and an ampicillin resistance gene to permit identification of transformed cells. More specifically, the base pUC19 vector is a small, high-copy number E. coli plasmid that is 2,686 base pairs in length. The base pUC19 plasmid contains: (1) the pMB1 replicon rep responsible for the replication of plasmid (the high-copy number of pUC plasmids is a result of the lack of the rop gene and a single point mutation in rep of pMB1); (2) the bla gene, coding for β-lactamase that confers resistance to ampicillin (the source being plasmid pBR322, but differing from pBR322 by two point mutations); (3) a region of the E. coli operon lac-containing CAP protein binding site, a promoter P_{lac}, a lac repressor binding site, and a 5′-terminal part of the lacZ gene encoding the N-terminal fragment of β-galactosidase.

To allow for the expression of protein under anoxic or microaerobic conditions, the pANX vector of the present invention includes a nitrate-inducible promoter in addition to the base pUC19 vector. Unless specifically differentiated, a nitrate-inducible promoter also encompasses nitrite and nitric oxide inducible promoters. In particular, the hmp promoter of the flavohemoglobin gene is used as the nitrate-inducible promoter. As described above, the E. coli flavohemoglobin gene (hmp) encodes a nitric oxide dioxygenase (NOD) that converts nitric oxide (NO) to nitrate, as described in Gardner, P. R., A. M. Gardner, L. A. Martin, and A. L. Salzman 1998 Nitric oxide dioxygenase: an enzymic function for flavohemoglobin. Proc Natl Acad Sci USA. 95:10378-10383, the disclosure of which is incorporated by reference herein in its entirety. NOD functions to detoxify NO generated as an intermediate in nitrogen metabolism or from exposure to the immune system. NOD (flavohemoglobin) is rapidly and strongly induced under both aerobic and anoxic exposure to NO, as described in Poole, R. K., M. F. Anjum, J. Membrillo-Hernández, S. O. Kim, M. N. Hughes, and V. Stewart. 1996. Nitric oxide, nitrite, and Fnr regulation of hmp (flavohemoglobin) gene expression in Escherichia coli K-12. Journal of Bacteriology, 178:5487-5492, the disclosure of which is incorporated by reference herein in its entirety.

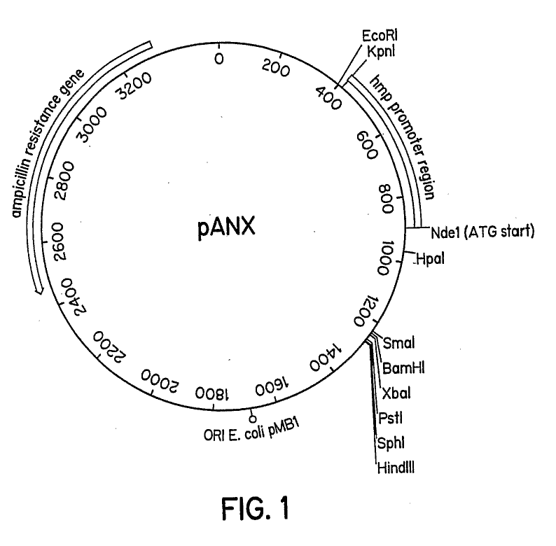

In vivo, the hmp promoter responds to anoxic nitrate and nitrite in addition to NO. (See Poole, R. K., et al.) Thus, the present invention includes a nitrate inducible expression plasmid using the upstream promoter region of hmp. Expression of oxygen-sensitive proteins would be facilitated by induction under conditions of low oxygen, or in the absence of oxygen. With reference to FIG. 1, in one embodiment the pANX vector of the present invention includes 812 bp of the E. coli flavohemoglobin upstream region and a portion of the NOD coding region cloned into pUC19. In alternate embodiments, the pANX vector may include less than 812 bp, such as a 244 bp sequence [SEQ. ID. NO. 6], and may include coding regions for genes of interest other than the region coding for NOD. Functionally, the DNA for the promoter sequence must include a nitrate-, nitrite-, or nitric oxide-inducible sequence, −35 and −10 bp RNA polymerase binding sites, and a ribosome-binding site. For example, all of these elements are present in the 244 bp sequence of SEQ. ID. NO. 6. In the pANX vector, the Nde1 site of pUC19 has been destroyed by filling in with Klenow fragment and religating the vector. Genes of interest are directionally cloned into pANX using a 5′ Nde1 site in the promoter region and any convenient 3′ polylinker site. A start methionine is encoded in the Nde1 site. Plasmids are maintained in the culture by ampicillin selection.

The 812 bp region in the pANX vector of FIG. 1 includes the hmp promoter (480 bp) and a partial NOD coding region (335 bp). The vector, in this illustrated embodiment, is constructed generally by subcloning the promoter and part of the NOD coding region into pUC19. There is a common ATG sequence coding for methionine that overlaps in the sequences of the promoter and the NOD coding region. In alternate embodiments, to express other proteins, the NOD coding region is replaced with a region coding for an alternate protein. The ATG sequence would be part of any other gene inserted in the vector. The ATG is part of the Nde1 restriction site, which is where other genes are inserted in-frame in the vector.

In other embodiments, the promoter region may include a smaller fragment than the 480 bp fragment discussed above. In general, any fragment that codes for inducibility via nitrate, nitrite, or nitric oxide would be sufficient. In order to produce such a fragment, one skilled in the art could first linearize the plasmid using Eco R1 restriction enzyme. Next, one would generate a nested series of deletions extending into the promoter, using, for example, a Bal31 exonuclease digestion. These shorter sequences would then be cloned into a reporter vector that lacks a promoter and assayed for inducibility of the reporter gene by nitrate, nitrite, or nitric oxide. Once the minimal length inducible sequence is determined, then nucleotides within this region would be randomly mutated by error-prone PCR (“EP-PCR”) to identify which nucleotides are essential and which are not essential for such inducibility. Briefly, sense and antisense primers are generated to the 5′ and 3′ ends of the DNA promoter segment. The average number of mutations per DNA fragment is controlled as a function of the number of EP-PCR doublings performed. The PCR fragments are subcloned into the reporter vector and screened for inducibility by nitrate, nitrite, or nitric oxide. These techniques are described in Current Protocols in Molecular Biology On Line (2004) FM Ausubel, ed. Wiley Intersciences, USA, which is incorporated by reference herein in its entirety.

Restriction endonuclease-digested DNA fragments are ligated into the pANX-cloning vector that has been cleaved with restriction endonucleases to create ends that will bind with the fragments containing the gene of interest. The ligation mixture is then used to transform an appropriate host, such as an E. coli strain. Colonies containing recombinant vectors are screened by PCR and confirmed with DNA sequencing.

In particular, an aerobic culture is used to start pANX expression cultures. Once the pANX plasmid containing the target gene is transformed into any common E. coli strain, the culture is grown aerobically to mid log phase in Luria Broth containing ampicillin. Luria Broth contains 10 g Bacto-Tryptone, 5 g yeast extract, 10 g NaCl prepared in 1 liter of deionized water, pH 7.2, and autoclaved for 60 minutes. Luria Broth is commercially available from Difco Laboratories of Detroit, Mich. Aerobic growth represses expression of the target protein and allows for a rapid growth rate of healthy cells.

To induce proteins from pANX, the starter culture is inoculated into nitrate-containing modified TB medium (Terrific Broth) (8 g Bacto-Tryptone, 24 g yeast extract, 5.04 g glycerol prepared in 900 ml deionized water; it is autoclaved 60 min., cooled, and 100 ml of filter sterilized 23.1 g/L KH₂PO₄ (anhydrous), 125.4 g/L K₂HPO₄ (anhydrous) supplemented with 10 mM nitrate, 150 μg/ml ampicillin is added in 1 L flasks (Pyrex # 4980) at an OD 0.01). Terrific Broth is commercially available from Difco Laboratories of Detroit, Mich. Because nitrate is used as an anoxic respiratory substrate, high levels of glucose are not required to support culture growth. It has been determined that glucose is actually inhibitory to protein induction from the pANX vector and thus the growth medium has been modified to contain less glucose by decreasing the amount of Bacto-Tryptone. Growth on glucose can also result in the production of toxic levels of acetate and other acids once oxygen saturation is reduced. Anoxic respiratory growth on nitrate eliminates this problem and avoids the addition of costly induction chemicals such as IPTG.

In particular, the cultures were grown at 37° C. with low shaking until saturation was reached. Growth and induction in static versus low aeration cultures was comparable (20-50% protein of interest). However, the final cell density was greater under microaerobic conditions (OD 2-4 for low aeration and OD 1.5-2 for static). Higher aeration (>200 RPM) resulted in little protein induction. The degree of aeration is directly proportional to the speed of shaking. Thus, increasing the speed of shaking in revolutions per minute increases the aeration. Cultures were harvested with minimal oxygen exposure and frozen. Cell pellets were lysed and the target protein was purified from the soluble protein fraction by standard means known to one skilled in the art.

The pANX vector was used to produce recombinant proteins that were sensitive to oxygen, as will be described further below and in the Examples.

Because of its cytotoxicity, an organism will metabolize NO. In E. coli, NO exposure induces metabolism in two ways: (1) an aerobic mechanism which induces NOD (encoded by the hmp gene); (2) and an anoxic mechanism, which induces NO reductase (containing two iron atoms and encoded by two genes: NorV and NorW genes). The NorV and NorW genes are transcriptionally controlled by an adjacent σ⁵⁴ dependent transcription regulator called NorR, which is a heme protein.

More specifically, the NorV/W genes encode a novel diiron-containing NO reductase. In E. coli, NO is detoxified by an O₂-independent NO reductase encoded by the NorV (flavorubredoxin) and NorW (NADH: flavorubredoxin oxidoreductase) genes arranged in a bicistronic operon and controlled by the adjacent σ⁵⁴-RNA polymerase-dependent regulator, NorR. E. coli NorR has been overexpressed and purified using the pANX vector. Based on its electronic absorption spectra, NorR is a heme protein. Ferrous NorR shows a Soret peak at 424 nm and α and β maxima at 557 nm and 531 nm, respectively. Oxidized NorR has a Soret peak at 412 nm and a broad band at 534 nm. These spectra are consistent with low-spin, hexacoordinate heme. NO binds ferrous NorR to form a five-coordinate nitrosyl ferrous heme, similar to the NO sensing soluble guanylate cyclase of mammalian cells. Although CO binds heme, only NO is physiologically active. While not being bound by any theory, the spectrum of CO bound NorR is similar to the CO sensing transcription factor CooA of Rhodospirillum rubrum. NorR contains a His111-X-Cys113 site reminiscent of the Cys75-X-His77 heme iron ligand-switch motif in CooA. It is believed that NorR senses NO through its heme and transduces this signal through structural changes resulting from the switch from hexacoordinate to pentacoordinate heme. This alters the conformation of the σ⁵⁴ interacting domain and activates transcription of the NorV/W genes. NorR orthologs are found adjacent to NorV/W in S. typhimurium, K. pneumoniae, and S. flexnerei, and adjacent to hmp in P. aeruginosa, V. cholera, and B. fungorum, suggesting a central role for NorR in bacterial NO stress responses.

In summary, the present invention includes a protein expression vector (pANX) and an expression system that is nitrate-inducible under anoxic conditions or conditions of low oxygen. The low oxygen growth conditions favor enhanced production and solubility of oxygen-sensitive proteins. Since the hmp promoter used in the pANX vector can be maximally induced under anoxic or microaerobic conditions, cells are grown rapidly under aerobic conditions, and then the promoter is induced by anoxic or microaerobic growth on nitrate. Thus, nitrate serves simultaneously as inducer, respiratory substrate, and energy source. Due to its low cost and lack of toxicity compared to IPTG, nitrate may provide an inexpensive alternative for the induction of hmp promoter mediated heterologous gene expression. In addition, induction under low oxygen eliminates the complications and expense of maintaining sufficient oxygenation in dense cultures of E. coli. Proteins, such as NOD, that constitutively produce damaging oxygen radicals, such as hydrogen peroxide or superoxide, can be expressed by this system. Proteins sensitive to oxygen damage are also successfully expressed from pANX. Although this procedure can be used for laboratory scale recombinant protein production, the production of other proteins of commercial interest will also be improved by this system and it can be adapted to industrial production using methods known to one skilled in the art. In particular, this is performed by an increase in the scale of production. At laboratory scale, 5 ml to 1 liter cultures may be used. In one embodiment, eight 1-liter batches may be used, from which a final yield of

50 mg to 1 g of greater than 90% pure protein may be expected.

As described above, pANX is a derivative of pUC19 in which a gene of interest, such as the NOD upstream region, has been inserted in the polylinker at the Sma1 site. Protein expression is driven by the E. coli flavohemoglobin promoter. The sequence of one embodiment of this pANX vector (coding for NOD) is shown in SEQ. ID. NO. 1.

The sequence of the base pUC19 vector is shown in SEQ. ID. NO. 2.

The sequence of the NOD coding region is shown in SEQ. ID. NO. 3.

The sequence of the polylinker region is shown in SEQ. ID. NO. 4.

The sequence of the hmp promoter region is shown in SEQ. ID. NO. 5.

The sequence of a 244 bp fragment of the hmp promoter region is shown in SEQ. ID. NO. 6.

The principles of the present invention will be more apparent with reference to the following Examples.

### Example 1

### Protein Expression Using the pANX Vector

The pANX vector, in this embodiment, is a derivative of pUC19 in which the NOD upstream region (812 bp Pvull to Sma1 fragment) was inserted in the polylinker at the Sma1 site. The Nde1 site of pUC19 was destroyed by filling in with Klenow fragment and religating. A start methionine was encoded in the Nde1 site in the NOD promoter. The NOD promoter can be induced to high level of expression with 10 mM nitrate in low aeration or anaerobic cultures.

Cultures of E. coli AB1157 with various protein coding regions subcloned in pANX were grown aerobically to OD₅₅₀ 0.6 in LB (Luria Broth) with 50 μg/ml ampicillin. The cultures were used to inoculate 900 ml of modified Terrific Broth to an optical density of 0.01 at 550 nm. Beef liver catalase (Roche) 260 U/ml final was added for protection of proteins sensitive to H₂O₂ damage. The cultures were grown overnight at 37° C. with slow shaking (˜100 revolutions per minute) in a rotary shaker-water bath. Cells were harvested by centrifugation and washed in 50 mM Tris-HCl, pH 8.0 at 4° C., 1 mM EDTA, with catalase 260 U/ml (Buffer A). The cell pellets were frozen on dry ice and stored at −70° C.

For analysis of soluble protein expression, cell pellets were thawed at 37° C. and lysed by sonication in chilled Buffer A at a cell volume:buffer ratio of about 1:4. The extracts were clarified by centrifugation >10,000×g for 30 min. Protein concentration was determined by the Bradford method using bovine serum albumin as the standard.

For gel analysis of protein induction, lysates were separated by SDS-PAGE on 8-16% gradient gels (Invitrogen) and proteins were visualized by Coomassie blue staining. Protein expression was quantitated by densitometric scanning of the gels.

### Example 2

### Induction of NOD

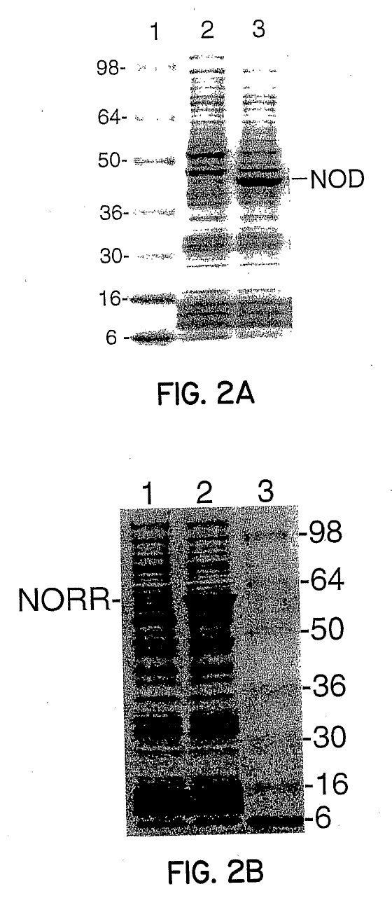

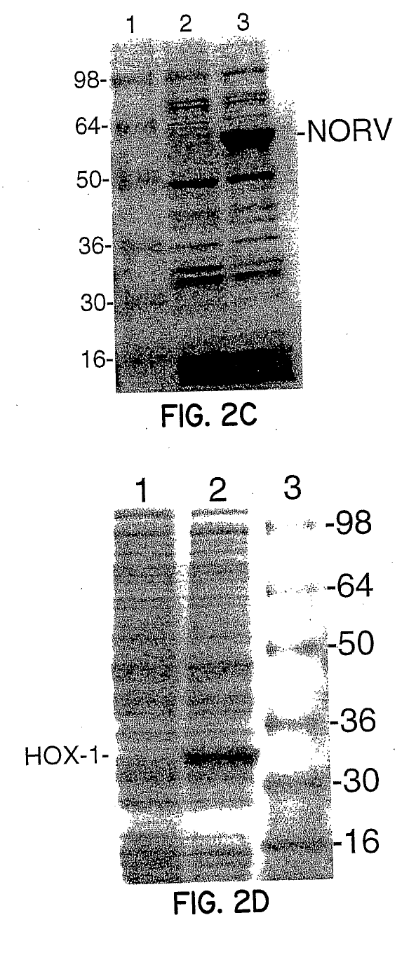

Referring to FIG. 2A, the coding region for NOD was subcloned in pANX, cultures were grown, and protein expression induced as described in Example 1. Induction and purification of NOD led to high yields, such as 50-200 mg of 90% pure protein from a 12-liter batch. NOD produced a low level of superoxide from bound oxygen and certain site-directed mutations in the NOD active site increased constitutive superoxide radical and hydrogen peroxide production to toxic levels. These proteins can only be efficiently produced under conditions of low aeration where superoxide radical and hydrogen peroxide production is limited.

### Example 3

### Induction of NorR

Referring to FIG. 2B, the coding region for NorR was subcloned in pANX, cultures were grown, and protein expression induced as described in Example 1. In the present invention, pANX was used to express the full-length NorR transcription factor in high yield, such as 50 mg of 90% pure protein from an 8-liter batch and in a soluble form (See FIG. 2B).

### Example 4

### Induction of NorV

The oxygen-sensitive diiron protein NorV was also expressed using pANX. The coding region for NorV was subcloned in pANX, cultures were grown, and protein expression induced as described in Example 1. Expression of NorV from pANX resulted in a high yield of soluble active protein (see FIG. 2C), such as 500 mg-1 g of 90% pure protein from an 8-liter batch. Loss of diiron resulted in inactivation of the NorV protein, when it was expressed under aerobic conditions. The NorV protein purified from nitrate induced low aeration cultures retained substantial iron and remained soluble.

### Example 5

### Induction of HOX-1

Referring to FIG. 2D, the coding region for human HOX-1 (heme oxygenase) was subcloned in pANX, cultures were grown, and protein expression induced as described in Example 1. Soluble HOX-1 was obtained in significant yield, such as 50-100 mg- of 90% pure protein from an 8-liter batch.

Other variations or embodiments of the invention will also be apparent to one of ordinary skill in the art from the above figures and descriptions. Thus, the forgoing embodiments are not to be construed as limiting the scope of this invention.

What is claimed is:

1. A vector to express recombinant proteins, comprising the nucleic acid sequence SEQ. ID. NO. 2, wherein said proteins are expressed under microaerobic or anoxic conditions.

2. The vector of claim 1, further comprising a promoter inducible by nitrate, nitrite, or nitric oxide.

3. The vector of claim 2, wherein said promoter comprises the nucleic acid sequence SEQ. ID. NO. 5.

4. The vector of claim 2, wherein said promoter comprises the nucleic acid sequence SEQ. ID. NO. 6.

5. The vector of claim 1, wherein said proteins are expressed in E. coli.

6. The vector of claim 5, wherein expression of said proteins is induced by a nitrate-, nitrite-, or nitric oxide-inducible hmp promoter of the flavohemoglobin gene of E. coli.

7. The vector of claim 1, wherein said proteins are oxygen-sensitive or oxygen-reactive proteins.

8. The vector of claim 7, wherein said oxygen-sensitive and oxygen-reactive proteins are selected from the group consisting of heme-oxygenase I, NorV/NorW nitric oxide reductase, C. albicans flavohemoglobin, a site-directed E. coli flavohemoglobin, and NorR transcription factor.

9. A vector to express recombinant proteins under microaerobic or anoxic conditions, said vector including a promoter that is inducible by nitrate, nitrite, or nitric oxide.

10. The vector of claim 9, wherein said promoter comprises the nucleic acid sequence SEQ. ID. NO. 5.

11. The vector of claim 9, wherein said promoter comprises the nucleic acid sequence SEQ. ID. NO. 6.

12. The vector of claim 9, wherein said proteins are expressed in E. coli.

13. The vector of claim 12, wherein said promoter is of the flavohemoglobin gene of E. coli.

14. The vector of claim 9, for use in expressing oxygen-sensitive or oxygen-reactive proteins.

15. The vector of claim 14, wherein said oxygen-sensitive and oxygen-reactive proteins are selected from the group consisting of heme-oxygenase I, NorV/NorW nitric oxide reductase, C. albicans flavohemoglobin, a site-directed E. coli flavohemoglobin mutant, flavohemoglobin, and NorR transcription factor.

16. A method of expressing a recombinant protein under anoxic or microaerobic conditions, comprising:
introducing a gene coding for a protein of interest into a vector capable of inducing expression of said protein of interest under anoxic or microaerobic conditions, wherein said vector comprises the nucleic acid sequence SEQ. ID. NO. 2; and
expressing said protein of interest in a host cell by inducing expression of said protein of interest with at least one of nitrate, nitrite, or nitric oxide.

17. The method of claim 16, said vector further comprising a promoter inducible by nitrate, nitrite, or nitric oxide, said promoter comprising the nucleic acid sequence SEQ. ID. NO. 5.

18. The method of claim 16, said vector further comprising a promoter inducible by nitrate, nitrite, or nitric oxide, said promoter comprising the nucleic acid sequence SEQ. ID. NO. 6.

19. A method of generating a nitrate inducible expression vector comprising:
inserting an expression-inducing fragment of an E. coli flavohemoglobin promoter into a pUC vector, thereby forming a pANX vector;
ligating into the pANX vector a DNA sequence encoding at least one of an oxygen-sensitive, oxygen-radical-generating, or oxygen-reactive protein;
transforming a bacterial host with the ligated pANX vector; and
selecting colonies that contain the ligated pANX vector.

20. The method of claim 19, further comprising reducing inclusion bodies in recombinant proteins by expressing recombinant proteins in said pANX vector under anoxic or microaerobic conditions, wherein said promoter is inducible by at least one of nitrate, nitrite, and nitric oxide.


SEQUENCE LISTING (6 sequences)

SEQ ID NO: 1 - DNA, 3503 bp, Homo sapiens
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccctgagaaccctgcgcctgcattacgcgcgggctggtgtagttttcggaggcgatcagttcgatgtgctcttcctgacgtactttttcctgctccatagcctgccacagttcggcatcataatcggcaatgttcatttcacgctttaacatccgcatctcctgactcagctaacaataaaatttttggcctttataggcggtcctgttggacaacggcgaacagtataaccgaatcattgtgcgataacaggtcttgacaaaggaatttacgcaaacgattaccttcaggctacgcaaggctttggagaataaagagcttgcaaccggaaacggatttcttttcaggtttgtgatgcaaatttttcacttcatcacattctttctgaaaaacaccaaagaaccatttacattgcagggctattttttataagatgcatttgagatacatcaattaagatgcaaaaaaaggaagaccatatgcttgacgctcaaaccatcgctacagtaaaagccaccatccctttactggtggaaacggggccaaagttaaccgcccatttctacgaccgtatgtttactcataacccagaactcaaagaaatttttaacatgagtaaccagcgtaatggcgatcaacgtgaagccctgtttaacgctattgccgcctacgccagtaatattgaaaacctgcctgcgctgctgccagcggtagaaaaaatcgcgcagaagcacaccagcttccagatcaaaccggaacagtacaacatcgtcggtgaacacctgttggcaacgctggacgaaatgttcagcccgggggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 2686 bp, Homo sapiens
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - DNA, 335 bp, Homo sapiens
atgcttgacgctcaaaccatcgctacagtaaaagccaccatccctttactggtggaaacggggccaaagttaaccgcccatttctacgaccgtatgtttactcataacccagaactcaaagaaatttttaacatgagtaaccagcgtaatggcgatcaacgtgaagccctgtttaacgctattgccgcctacgccagtaatattgaaaacctgcctgcgctgctgccagcggtagaaaaaatcgcgcagaagcacaccagcttccagatcaaaccggaacagtacaacatcgtcggtgaacacctgttggcaacgctggacgaaatgttcagccc

SEQ ID NO: 4 - DNA, 44 bp, Homo sapiens
cccgggggggatcctctagagtcgacctgcaggcatgcaagctt

SEQ ID NO: 5 - DNA, 480 bp, Homo sapiens
ctgagaaccctgcgcctgcattacgcgcgggctggtgtagttttcggaggcgatcagttcgatgtgctcttcctgacgtactttttcctgctccatagcctgccacagttcggcatcataatcggcaatgttcatttcacgctttaacatccgcatctcctgactcagctaacaataaaatttttggcctttataggcggtcctgttggacaacggcgaacagtataaccgaatcattgtgcgataacaggtcttgacaaaggaatttacgcaaacgattaccttcaggctacgcaaggctttggagaataaagagcttgcaaccggaaacggatttcttttcaggtttgtgatgcaaatttttcacttcatcacattctttctgaaaaacaccaaagaaccatttacattgcagggctattttttataagatgcatttgagatacatcaattaagatgcaaaaaaaggaagaccatatg

SEQ ID NO: 6 - DNA, 244 bp, Homo sapiens
ttgtgcgataacaggtcttgacaaaggaatttacgcaaacgattaccttcaggctacgcaaggctttggagaataaagagcttgcaaccggaaacggatttcttttcaggtttgtgatgcaaatttttcacttcatcacattctttctgaaaaacaccaaagaaccatttacattgcagggctattttttataagatgcatttgagatacatcaattaagatgcaaaaaaaggaagaccatatg